(19) United States
(12) Patent Application Publication
(10) Pub. No.: US 2004/0143009 A1
(43) Pub. Date: Jul. 22, 2004

(54) Amines, uses thereof

(75) Inventors: Maria Dalko, Gif S/Yvette (FR); Lionel Breton, Versailles (FR); Christophe Boulle, Lagny S/Marne (FR)

Correspondence Address: OBLON, SPIVAK, MCCLELLAND, MAIER & NEUSTADT, P.C., 1940 DUKE STREET, ALEXANDRIA, VA, 22314, US

(73) Assignee: L'OREAL, Paris (FR)

(21) Appl. No.: 10/701,466

(22) Filed: Nov. 6, 2003

Related U.S. Application Data
(60) Provisional application No. 60/428,744, filed on Nov. 25, 2002.

(30) Foreign Application Priority Data
Nov. 5, 2002 (FR) 0214321

(51) Int. Cl. A61K 31/277; A61K 31/137; A61K 31/195; A61K 31/165; A61K 31/17

(52) U.S. Cl. 514/524; 514/567; 514/619; 514/649; 514/597; 558/418; 562/440; 564/51; 564/164; 564/336

(57) ABSTRACT

The present invention relates to novel secondary or tertiary amines useful for smoothing wrinkles and fine lines, in particular expression wrinkles and fine lines.

### REFERENCE TO PRIOR APPLICATIONS

This application claims priority to U.S. provisional application 60/428,744 filed Nov. 25, 2002, and to French patent application 0214321 filed Nov. 15, 2002, both incorporated herein by reference.

### FIELD OF THE INVENTION

The present invention relates to novel secondary and tertiary amines, and to a composition, preferably one suitable for topical application to the skin, comprising at least one such amine in a physiologically acceptable medium.

The invention also relates to the use of such amines in a composition suitable for topical application to the skin, as agent intended for smoothing wrinkles and fine lines, in particular expression wrinkles and fine lines.

Additional advantages and other features of the present invention will be set forth in part in the description that follows and in part will become apparent to those having ordinary skill in the art upon examination of the following or may be learned from the practice of the present invention. The advantages of the present invention may be realized and obtained as particularly pointed out in the appended claims. As will be realized, the present invention is capable of other and different embodiments, and its several details are capable of modifications in various obvious respects, all without departing from the present invention. The description is to be regarded as illustrative in nature, and not as restrictive.

### BACKGROUND OF THE INVENTION

Women, or even men, nowadays tend to want to look young for as long as possible and consequently seek to attenuate the signs of skin ageing, which manifest themselves in particular in wrinkles and fine lines. In this regard, advertisements and fashion promote products intended to maintain for as long as possible a radiant skin without wrinkles, which is the sign of a young skin, all the more so since the physical appearance acts on the mind and on the morale.

Up until now, wrinkles and fine lines were treated with the aid of cosmetic products containing active agents acting on the skin, for example by moisturizing it or by enhancing its cellular renewal or by promoting the synthesis, or preventing the degradation, of the supporting fibres which constitute the skin tissue.

Although these treatments make it possible to act on the wrinkles and fine lines caused by chronologic or intrinsic ageing, and on those caused by photoageing, they have no effect on expression wrinkles and fine lines, which require action on the muscle contractile component of the wrinkles present in the skin.

Up until now, the only means commonly used for acting on expression wrinkles is the botulinum toxin which is in particular injected into the wrinkles of the glabella which are intersuperciliary wrinkles (see J. D. Carruters et al., J. Dermatol. Surg. Oncol. 1992, 18, pp. 17-21). Dermatologists have also sometimes had recourse to implants of hyaluronic acid or of polylactic acid.

These techniques however have the disadvantage of requiring recourse to be had to a practitioner.

In the perspective of providing solutions which are simpler to use than these medical techniques, the present assignee has provided various compounds which can offer a muscle-relaxing effect when they are topically applied to the skin, thus making it possible to act by another route on expression wrinkles. Among these compounds, there may be mentioned in particular antagonists of the receptors associated with calcium channels (FR-2 793 681), and in particular manganese and its salts (FR-2 809 005) and alverine (FR-2 798 590); and agonists of the receptors associated with the chlorine channels, including glycine (EP-0 704 210) and certain extracts of Iris pallida (FR-2 746 641).

The need however remains to have available compounds which are effective for smoothing or attenuating expression wrinkles and fine lines.

### BRIEF DESCRIPTION OF THE DRAWINGS

FIG. 1 depicts the reaction scheme for a compound prepared in the Example Section.

FIG. 2 depicts the reaction scheme for a compound prepared in the Example Section.

FIG. 3 depicts the reaction scheme for a compound prepared in the Example Section.

### DETAILED DESCRIPTION OF THE PREFERRED EMBODIMENTS

Now, the inventors have discovered, surprisingly, that certain secondary or tertiary amines satisfy the above-stated need.

Secondary and tertiary amines are known from the document EP-1 090 630 which have the property of increasing the synthesis of collagen by the fibroblasts and of moisturizing the skin and are, because of this, useful against dry skin and atopic dermatitis, but also against wrinkles. Likewise the document EP-0 691 327 discloses a very vast family of mono-, di- or trisubstituted amines described as being effective for smoothing wrinkles. There is no suggestion that the amines disclosed in these documents can have any effect on expression wrinkles and fine lines, unlike the amines which are the subject of the present invention which constitute a selection among the very vast family of amines disclosed in EP-1 090 630 and EP-0 691 327.

Certain amines which are di- or trisubstituted with at least two chains each carrying at least one hydroxyl group are moreover known from the document WO 93/05763. These amines increase the differentiation of the keratinocytes, limit UV-induced thickening of the epidermis and are useful for preventing and treating the wrinkles induced by UVB radiation. There is no suggestion that these amines, which are different from those which are the subject of the present invention in the sense that they do not comprise a phenyl group, have any effect on expression wrinkles and fine lines.

Finally, a family of calcium antagonists consisting of disubstituted arylalkylamines, which are in particular capable of modulating the differentiation of the keratinocytes, is known from WO 97/37967. A family of ethanolamines having a structure similar to those above, which are useful as modulators of adrenergic receptors, is also known from the publication by Shuker A. J. et al., The Application of High-Throughput Synthesis And Purification To The Preparation Of Ethanolamines, Tetrahedron Letters, Vol. 38, No. 35, pp. 6149-6152 (1997). In these two families of compounds, the phenyl group is separated from the nitrogen atom by a branched chain, which distinguishes them from those which are the subject of the present invention. In addition, there is no suggestion that these compounds have any effect on wrinkles, a fortiori on expression wrinkles.

The inventors have now discovered that by selecting certain secondary and tertiary amines having a simple structure, it was possible to obtain cosmetic compositions effective for smoothing expression wrinkles and fine lines.

The use of alverine, which is a trisubstituted amine, as a muscle-relaxant intended for smoothing expression wrinkles has admittedly been previously described by the applicant. However, unlike the compounds which are the subject of the present invention, alverine is an amine substituted with two aralkyl chains. Now, it was not obvious for the muscle-relaxant activity of alverine to be increased by substituting in its molecule an alkyl chain for an aralkyl chain.

The subject of the present invention is novel secondary or tertiary amines of formula (I):

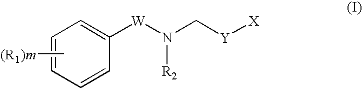

in which:

R₁ denotes a saturated or unsaturated, linear or branched C₁-C₈ alkyl group, or a group —CN, —OR₁₁, —SR₁₁, —NR₁₁R₁₂, —COR₁₁, —COOR₁₁, —CONR₁₁R₁₂, —NR₁₁—CO—R₁₂, —NR₁₁—CO—NR₁₂R₁₃ or —CF₃ or a halogen atom,

where R₁₁, R₁₂ and R₁₃ independently denote a hydrogen atom or a linear or branched C₁-C₄ alkyl group, or an aryl group optionally substituted with a group —OR, —NRR′, —COOR or CF₃,

where R and R′ independently denote a hydrogen atom or a linear or branched C₁-C₄ alkyl group,

R₂ denotes a hydrogen atom or an unsubstituted, saturated or unsaturated, linear or branched C₁-C₁₂ alkyl group,

W is an unsubstituted, linear C₂-C₄ alkylene or alkenylene chain,

X is a group —OR₁₁ or —NR₁₁R₁₂, where R₁₁ and R₁₂ have the meaning indicated above,

Y denotes an unsubstituted, linear or branched C₁₁-C₂₀ alkylene or alkenylene chain,

m is an integer between 0 and 5,

it being understood that when m is not zero, the groups R₁ may be identical or different,

and their addition salts with an acid and its isomers and stereo-isomers.

In formula (I), the alkyl groups may be chosen, depending on the case, from the groups: methyl, ethyl, n-propyl, isopropyl, n-butyl, isobutyl, tert-butyl, pentyl, hexyl, heptyl, octyl, nonyl, decyl, undecyl and dodecyl.

For its part, the aryl group may be a phenyl group.

The C₂-C₄ alkylene or alkenylene chain is in particular an ethylene, trimethylene, tetramethylene, vinylene or propenylene chain.

Y preferably denotes an unsubstituted di(C₅-C₇) alkyl ethylene or di (C₅-C₇) dialkyl pentylene chain and more particularly a dipentylethylene chain.

The halogen atom may preferably be a fluorine, chlorine or bromine atom.

Salts of the compound of formula (I) include, for example, the salts obtained by addition of the compound of formula (I) with an inorganic acid, chosen in particular from hydrochloric, sulphuric, nitric and phosphoric acids, or with an organic acid, chosen in particular from succinic, fumaric, lactic, glycolic, citric and tartaric acids.

According to a preferred embodiment of the invention, the compound of formula (I) is such that at least one of the following conditions is satisfied:

m=0 or 1,

R₁ is a group —OR₁₁ where R₁₁ is a hydrogen atom or a linear or branched C₁-C₄ alkyl group,

R₂ is a hydrogen atom or an unsubstituted, saturated, linear or branched C₁-C₆ alkyl group,

W is an unsubstituted, linear C₂-C₄ alkylene or alkenylene chain,

X is a group —OR₁₁, where R₁₁ is a hydrogen atom or a linear or branched C₁-C₄ alkyl group,

Y is an unsubstituted, branched C₁₁-C₁₆ alkylene chain.

Still more preferably, the compound of formula (I) is such that:

m=0 or 1,

R₁ is a group —OH or —OCH₃,

R₂ is a hydrogen atom or an ethyl group,

W is a trimethylene or propenylene chain,

X is a group —OH,

Y is an unsubstituted di(C₅-C₇)alkyl ethylene or di(C₅-C₇)dialkyl pentylene chain.

According to a particularly preferred embodiment of the invention, the compound of formula (I) is such that: m=0; R₂ is an ethyl group; X═OH; Y is a dipentylethylene chain; and W is a trimethylene chain.

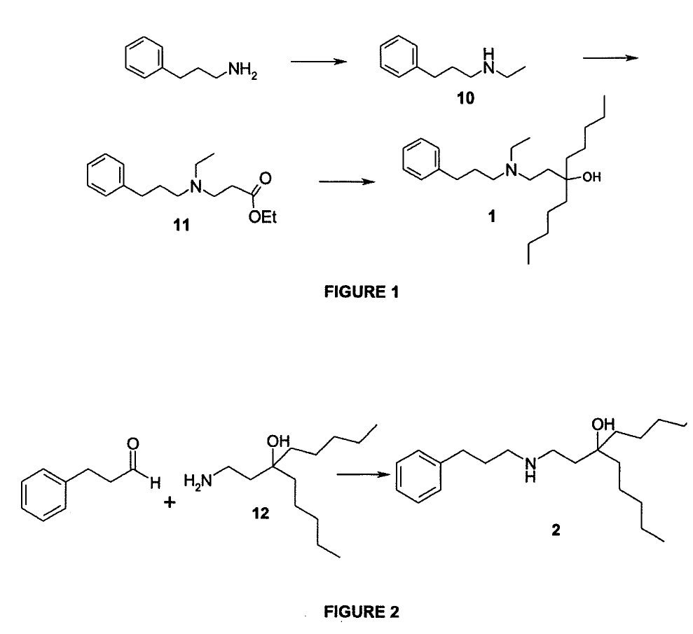

The amines of formula (I) may be prepared according to methods similar to the reaction schemes presented in the accompanying FIGS. 1 to 3 and to those described in Examples 1 to 9 below.

The invention also relates to a composition, suitable for topical application to the skin, comprising, in a physiologically acceptable medium, at least one compound of formula (I) in which W may additionally denote a methylene chain, or an addition salt with an acid or an isomer or stereo-isomer of this compound.

The quantity of amine of formula (I) which can be used according to the invention of course depends on the desired effect and can therefore vary to a great extent. To give an order of magnitude, it is possible to use this amine in a quantity representing from 0.01% to 10% of the total weight of the composition, preferably in a quantity representing from 0.05% to 5% of the total weight of the composition, more preferably in a quantity representing from 0.1% to 2% of the total weight of the composition.

The composition according to the invention is preferably suitable for topical application to the skin and it therefore preferably contains a physiologically acceptable medium, that is to say which is compatible with the skin and possibly with its superficial body growths (eyelashes, nails, hair) and/or the mucous membranes.

This composition may be provided in any form, including those galenic forms normally used in the cosmetic field, and it may be in particular in the form of an optionally gelled oily solution, a two-phase lotion-type dispersion, an emulsion obtained by dispersing a fatty phase in an aqueous phase (O/W) or conversely (W/O), or a triple emulsion (W/O/W or O/W/O) or a vesicular dispersion of the ionic and/or nonionic type. These compositions are prepared according to the customary methods. It is preferable to use according to this invention a composition in the form of an oil-in-water emulsion.

This composition may be fluid to a greater or lesser degree and may have the appearance of a white or coloured cream, an ointment, a milk, a lotion, a serum, a paste, a mousse. It may be optionally applied in the form of an aerosol. It may also be provided in solid form, in particular in stick form. It may be used as a care product and/or as a makeup product for the skin.

The composition used according to the invention may also contain adjuvants, such as the usual adjuvants in the cosmetic field, such as hydrophilic or lipophilic gelling agents, hydrophilic or lypophilic active agents, preservatives, antioxidants, solvents, perfumes, fillers, screening agents, pigments, odour absorbers and colouring substances. The quantities of these various adjuvants may be those conventionally used in the field considered, and for example from 0.01 to 20% of the total weight of the composition. These adjuvants, depending on their nature, may be introduced into the fatty phase, into the aqueous phase or into the lipid vesicles. In any case, these adjuvants, and their proportions, will preferably be chosen so as not to adversely affect the desired properties of the amines of formula (I) according to the invention.

When the composition used according to the invention is an emulsion, the proportion of the fatty phase may preferably range from 5 to 80% by weight, and more preferably from 5 to 50% by weight relative to the total weight of the composition. The oils, the emulsifiers and the coemulsifiers used in the composition in the form of an emulsion may be chosen from those conventionally used in the field considered. The emulsifier and the coemulsifier are preferably present in the composition in a proportion ranging from 0.3 to 30% by weight, and more preferably from 0.5 to 20% by weight relative to the total weight of the composition.

As oils which can be used in the invention, there may be mentioned as being included mineral oils (liquid paraffin), oils of plant origin (avocado oil, soya-bean oil), oils of animal origin (lanolin), synthetic oils (perhydrosqualene), silicone oils (cyclomethicone) and fluorinated oils (perfluoropolyethers). It is also possible to use, as fatty substances, fatty alcohols (cetyl alcohol), fatty acids, waxes (carnauba wax, ozokerite).

As emulsifiers and coemulsifiers which can be used in the invention, there may be mentioned for example fatty acid esters of polyethylene glycol such as PEG-100 stearate, and fatty acid esters of glycerin such as glyceryl stearate.

Useful hydrophilic gelling agents include, in particular, carboxyvinyl polymers (carbomer), acrylic copolymers such as copolymers of acrylates/alkyl acrylates, polyacrylamides, polysaccharides, natural gums and clays, and, as lipophilic gelling agents, there may be mentioned modified clays such as bentones, metal salts of fatty acids, hydrophobic silica and polyethylenes.

As active agents, it can be advantageous to introduce into the composition used according to the invention at least one compound chosen from: desquamating agents; moisturizing agents; depigmenting or propigmenting agents; antiglycation agents; NO-synthase inhibitors; agents stimulating the synthesis of dermal or epidermal macromolecules and/or preventing their degradation; agents stimulating the proliferation of fibroblasts and/or of the keratinocytes or stimulating the differentiation of the keratinocytes; other muscle-relaxing agents; tightening agents; antipollution and/or anti-radical agents; agents acting on the microcirculation; agents acting on the energy metabolism of the cells; and mixtures thereof.

The composition according to the invention may also contain UVA and/or UVB screening agents in the form of organic or inorganic compounds, the latter being optionally coated in order to make them hydrophobic.

The organic photoprotective agents more particularly preferred are chosen from the following compounds (identified under their CTFA name): ethylhexyl salicylate, ethylhexyl methoxycinnamate, octocrylene, phenylbenzimidazole sulphonic acid, benzophenone-3, benzophenone-4, benzophenone-5,4-methylbenzylidene camphor, terephthalylidene dicamphor sulphonic acid, disodium phenyl dibenzimidazole tetrasulphonate, 2,4,6-tris-(diisobutyl 4′-aminobenzalmalonate)-s-triazine, anisotriazine, ethylhexyl triazone, diethylhexyl butamido triazone, methylene bis-benzotriazolyl tetramethylbutylphenol, drometrizole trisiloxane, 1,1-dicarboxy (2,2′-dimethylpropyl)-4,4-diphenylbutadiene, and mixtures thereof.

The inorganic photoprotective agents may be chosen from pigments or alternatively nanopigments (mean primary particle size: generally between 5 nm and 100 nm, preferably between 10 nm and 50 nm) of metal oxides which are coated or uncoated, such as for example nanopigments of titanium oxide (amorphous or crystallized in rutile and/or anatase form), of iron oxide, of zinc oxide, of zirconium oxide or of cerium oxide which are all UV photoprotective agents well known per se. Conventional coating agents are moreover alumina and/or aluminium stearate.

As will be demonstrated in the examples below, the inventors have identified a muscle-relaxing effect of the amines of formula (I) according to the invention, which makes it possible to use them, more particularly in the smoothing of expression wrinkles and fine lines.

The subject of the invention is therefore also the cosmetic use of at least one amine of formula (I) as defined above, in which W may additionally denote a methylene chain, in a composition suitable for topical application to the skin, as agent intended for, and in an amount sufficient for, smoothing wrinkles and fine lines, in particular expression wrinkles and fine lines.

Its subject is also a method for the cosmetic treatment of a wrinkled skin, comprising the topical application to the said skin of a composition as defined above in which W may additionally denote a methylene chain.

The composition according to the invention is advantageously intended to be applied to the areas of the face or of the forehead marked by expression wrinkles and fine lines, and/or to people having expression wrinkles and fine lines.

The wrinkles and fine lines in question are preferably those arranged radially around the mouth and/or the eyes, in particular crow's-foot wrinkles, and/or situated on the forehead, in particular the so-called lion's wrinkle, situated on the glabella, in the inter-superciliary space, and/or arranged horizontally on the forehead.

The invention will now be illustrated by the following nonlimiting examples.

### EXAMPLES

### Example 1

### Synthesis of 6-[2-{Ethyl-(3-Phenylpropyl)-Amino}Ethyl]Undecan-6-ol (Compound 1)

This compound is prepared according to the reaction scheme presented in FIG. 1.

a) Ethyl-(3-Phenylpropyl)Amine

1 equivalent of commercial 3-phenylpropylamine (36.98 mmol; 5.3 ml), 1 equivalent of acetaldehyde (36.98 mmol; 2.1 ml) and 25 ml of ethanol are mixed and heated at 50° C. for 3 hours. 1.4 equivalents of sodium borohydride (9.320 mmol; 353 mg) are then added with small spatulas and the medium is stirred at room temperature for 2 hours. The reaction medium is concentrated and then taken up in water; the organic phase is extracted with 2 fractions of 20 ml of dichloromethane, and then the organic phase is dried over anhydrous sodium sulphate and concentrated to dryness.

6.0 g of compound 10 are thus obtained in the form of a yellow oil (yield: 99%).

b) Ethyl 3-[Ethyl-(3-Phenylpropyl)Amino]Propionate

1.5 equivalents of potassium carbonate (3.675 mmol; 507 mg) and 2 equivalents of ethyl 3-bromopropionate (4.900 mmol; 0.57 ml) are added to 1 equivalent of ethyl-(3-phenylpropyl)amine (2.450 mmol; 400 mg) solubilized in 10 ml of dimethylformamide, and then the medium is heated at 50° C., with stirring, for 10 hours. The reaction medium is diluted in a large volume of dichloromethane, washed with a saturated sodium hydrogen carbonate solution, dried over anhydrous sodium sulphate, filtered and concentrated to dryness. The oil obtained is purified on a silica column (dichloromethane/methanol).

100 mg of compound 11 are obtained in the form of a yellow oil (yield=17%).

c) 6-[2-(3-Phenylpropylamino)Ethyl]Undecan-6-ol

2 equivalents of magnesium chips (15.2 mmol; 370 mg) are poured into a set-up dried beforehand and under an inert atmosphere and they are covered with anhydrous ether. An iodine crystal and a few drops of bromopentane are added; the medium is gently stirred and heated. When the reaction starts, the remainder of bromopentane (2 equivalents; 15.2 mmol; 1.9 ml) diluted in anhydrous ether is added. The medium is kept stirred at room temperature for 1 hour. When all the magnesium has dissolved, the reaction medium is cooled to 0° C., and 1 equivalent of ethyl 3-[ethyl-(3-phenylpropyl)-amino]propionate (7.6 mmol; 2 g) diluted in anhydrous ether is added dropwise. The reaction medium is allowed to return to room temperature and it is stirred for 15 minutes. Water is added to the reaction medium, followed by a 0.1 M ammonium chloride solution. The organic phase is extracted with dichloromethane, it is washed with a 0.1 M NaHCO₃ solution, it is dried over anhydrous sodium sulphate and it is concentrated to dryness. The product is purified on a silica column (2% methanol/98% dichloromethane).

200 mg of compound 1 are obtained in the form of a yellow oil (yield=8%).

### Example 2

### Synthesis of 6-{2-(3-Phenylpropyl)Amino-Ethyl}Undecan-6-ol (Compound 2)

This compound is synthesized according to the reaction scheme presented in FIG. 2.

1 equivalent of 3-phenylpropionaldehyde (6.657 mmol; 0.88 ml) is added dropwise to 1 equivalent of 6-(2-aminoethyl)undecan-6-ol (6.657 mmol; 1.43 g) 12 solubilized in 5 ml of ethanol. After stirring at room temperature for 3 hours, 1.4 equivalents of sodium borohydride (9.320 mmol; 353 mg) are added with small spatulas and the medium is again stirred at room temperature for 2 hours. The reaction medium is dried and the oil obtained is taken up in water. The organic phase is extracted with 2 fractions of 20 ml of dichloromethane, dried over anhydrous sodium sulphate and concentrated to dryness.

1.8 g of compound 2 are obtained in the form of a yellow oil (yield=81%).

### Example 3

### Synthesis of 6-(2-{Ethyl-[3-(4-Methoxy-Phenyl)Propyl]Amino}Ethyl}Undecan-6-ol (Compound 3)

This compound is prepared according to a reaction scheme similar to that presented in FIG. 1.

Ethyl-(3-p-methoxyphenylpropyl)amine is thus prepared from (3-p-methoxyphenylpropyl)amine in a manner similar to compound 10 and ethyl 3-[ethyl-(3-p-methoxyphenylpropyl)amino]propionate in a manner similar to compound 11. The ester obtained is then subjected to the same reactions as compound 11 in order to obtain compound 3.

250 mg of compound 3 are obtained in the form of a yellow oil (yield: 8%).

### Example 4

### Synthesis of 6-(2-[3-(4-Methoxyphenyl)-Propylamino]Ethyl}Undecan-6-ol (Compound 4)

This compound is prepared according to a reaction scheme similar to that presented in FIG. 2, except that 3-(4-methoxyphenyl)propionaldehyde is substituted for 3-phenylpropionaldehyde.

1.9 g of compound 4 are thus obtained in the form of a yellow oil (yield: 79%).

### Example 5

### Synthesis of 6-{3-[Ethyl-(3-Phenylpropyl)-Amino]Propyl}Undecan-6-ol (Compound 5)

Compound 5 is prepared in a manner similar to compound 1, except that ethyl 3-bromopropionate is replaced by methyl 4-bromobutyrate.

200 mg of compound 5 are obtained in the form of a yellow oil (yield: 7%).

### Example 6

### Synthesis of 6-[3-(3-Phenylpropylamino)-Propyl]Undecan-6-ol (Compound 6)

Compound 6 is prepared in a manner similar to compound 2, except that reagent 12 is replaced by 6-(3-aminopropyl)undecan-6-ol.

1.9 g of compound 6 are obtained in the form of an oil (yield: 82%).

### Example 7

### Synthesis of 6-(3-{Ethyl-[3-(4-Methoxy-Phenyl)Propyl]Amino}Propyl)Undecan-6-ol (Compound 7)

Compound 7 is prepared in a manner similar to compound 1, using as starting material 3-(4-methoxy)phenylpropylamine and replacing ethyl 3-bromopropionate by ethyl 3-bromobutyrate.

230 mg of compound 7 are obtained in the form of a yellow oil (yield: 7%).

### Example 8

### Synthesis of 6-{3-[3-(4-Methoxyphenyl)-Propylamino]Propyl}Undecan-6-ol (Compound 8)

Compound 8 is prepared in a manner similar to compound 4, except that the starting aldehyde is reacted with 6-(3-aminopropyl)undecan-6-ol.

1.9 g of compound 8 are obtained in the form of an oil (yield: 76%).

### Example 9

### Synthesis of 6-{2-[Ethyl-(3-Phenylallyl)-Amino]Ethyl}Undecan-6-ol (Compound 9)

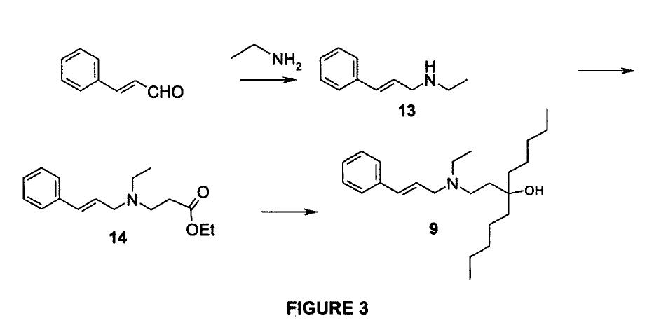

This compound is prepared according to the reaction scheme illustrated in FIG. 3.

a) Ethyl-(3-Phenylallyl)Amine

1 equivalent of ethylamine at 70% in water (0.038 mmol; 2.44 g) is added dropwise to 1 equivalent of cinnamaldehyde (0.038 mmol; 5 g) mixed with 10 ml of ethanol. The medium is stirred at room temperature for 1 hour, and then 2 equivalents of sodium borohydride (0.076 mmol; 2.86 g) are added with small spatulas (exothermic reaction) and the medium is kept stirred at room temperature for 1 hour. A large quantity of water is then added and the organic phase is extracted with dichloromethane, it is dried over anhydrous sodium sulphate and it is concentrated to dryness.

5.51 g of compound 13 are obtained in the form of a yellow oil (yield: 90%).

b) Ethyl 3-[Ethyl-(3-Phenylallyl)Amino]Propionate

1 equivalent of ethyl-(3-phenylallyl)amine 13 (0.031 mmol; 5 g), 1 equivalent of potassium carbonate (0.031 mmol; 4.3 g) and 25 ml of dimethylformamide are mixed in a 50 ml three-necked flask. 1 equivalent of ethyl 3-bromopropionate (0.031 mmol; 3.4 ml) is then added dropwise and the medium is stirred at room temperature for 5 hours. The reaction medium is diluted in dichloromethane and washed with a saturated sodium hydrogen carbonate solution. The organic phase is dried over anhydrous sodium sulphate and concentrated to dryness.

6.5 g of compound 14 are obtained in the form of a yellow oil (yield: 80%).

c) 6-{2-[Ethyl-(3-Phenylallyl)Amino]Ethyl}undecan-6-ol

2 equivalents of magnesium chips (0.023 mmol; 560 mg) are added to the set-up dried beforehand and they are covered with anhydrous ether. An iodine crystal and a few drops of pure bromopentane are added; the medium is slowly stirred and heated a little if necessary. When the reaction has started, the remainder of the bromopentane (2 equivalents; 0.023 mmol; 2.86 ml) diluted in anhydrous ether is added (exothermic reaction). The medium is kept stirred at room temperature for 1 hour approximately. When all the magnesium has dissolved, the reaction medium is cooled to 0° C. and 1 equivalent of ethyl 3-[ethyl-(3-phenylallyl)amino]propionate (0.012 mmol; 3 g) 14 diluted in anhydrous ether is added dropwise; the reaction medium is allowed to return to room temperature and the medium is stirred for 1 quarter of an hour. Water is added to the reaction medium, followed by a 0.1 M ammonium chloride solution. The organic phase is extracted with dichloromethane, it is dried over anhydrous sodium sulphate and it is concentrated to dryness. The product is purified on a silica column (dichloromethane/methanol).

227 mg of compound 9 are obtained in the form of a yellow oil (yield: 6%).

### Example 10

### Demonstration of the Muscle-Relaxing Effect of the Amines According to the Invention

a) Nerve/Muscle Junction Model

Compound 1 was tested on a nerve/muscle (motor plate) junction model obtained in a rat-isolated phrenic nerve/diaphragm (striated muscle) preparation (Pollard B. J. et al., Br. J. Anaesth., 1988, 61, 419-424).

This test is predictive of an antiwrinkle effect, as was verified by the applicant on diazepam, which is both active in vivo in humans and in this test at 10⁻⁴ M.

The phrenic nerve and the diaphragm are carefully isolated and placed in a 50 ml tank filled with survival fluid (Krebs Henseleit fluid) kept at a temperature of 37° C. and oxygenated with an oxygen/CO₂ (95/5) mixture.

Variations in the tension of the diaphragm are recorded with an initial preload of several grams.

After a relaxation period of 30 min, the diaphragm is indirectly stimulated by means of the phrenic nerve.

On each preparation, the effect of the test compound was evaluated on contractions induced by indirect stimulation via stimulation on the phrenic nerve (0.1 to 0.5 volts, 0.3 ms, 0.1 Hz). Alverine, known as a muscle-relaxing compound with antiwrinkle effect according to application FR-2 798 590, is used as control.

The results obtained are presented in the table below:

| | | % INHIBITION OF |
|---|---|---|
| PRODUCT | CONCENTRATION | CONTRACTIONS |
| Alverine | 10⁻⁴ M | 100% |
| Compound 1 | 10⁻⁴ M | 100% |

b) Test on Calcium Channels

The test measures the capacity of a product to inhibit, by competition, the binding of L-type, Ver-pamil a type, calcium channel agonists. These channels were identified in human fibroblasts (Baumgarten LB et al., (1992), J. Biol. Chem., 267, 10524-10530 and Chen CF et al., (1988), Science, 239, 1024-1026).

The studies are carried out using rat cerebral cortex homogenates (isolated membranes having at their surface in particular L-type calcium channels).

The experimental conditions according to the protocol described by Reynolds I. J. et al., 1986, J. Pharmacol. Exp. Ther., 237, p. 731, are presented in Table 1 below:

TABLE 1

| | | | Non- | | |
|---|---|---|---|---|---|
| Test | Ligand | Conc. | specific | Incubation | Detection |
| Ca²⁺ channel | [³H] (−) | 0.5 nM | D 600 | 60 min./ | Scintillation |
| (L, verapamil | D 888 | | (10 μM) | 22° C. | counting |
| site) | | | | | |

D888 and D600 are reference molecules specific for the L-type calcium channels, verapamil site.

The specific binding of a ligand (labelled D888) to the receptors (L-type calcium channels, verapamil site) is defined as the difference between the total binding and the nonspecific binding determined in the presence of an excess of nonradioactive ligand. The results are expressed as a percentage of the control specific binding and as a percentage inhibition of this binding in the presence of the test compounds.

The IC₅₀ (concentration inhibiting 50% of the control specific binding) and the Hill coefficient (nH) are calculated for the test compounds from competition curves according to a nonlinear regression model. These parameters are obtained by the Hill equation “curve fitting”.

Two compounds were tested: alverine, which is a muscle-relaxing agent inhibiting calcium channels according to application FR-2 798 590, and compound 1 described above (Example 1), each at the concentrations of 1 μM and 100 μM. The measurements are carried out in duplicate. The reference molecule (D600) is tested in parallel at eight concentrations and in duplicate in order to obtain a competition curve which makes it possible to validate this test.

The results obtained are presented in Table 2 below:

TABLE 2

| Compound | IC₅₀ |
|---|---|
| Alverine | 693 nM |
| Compound 1 | 325 nM |

Compound 1 according to the invention is therefore a better calcium channel inhibitor than alverine.

c) Conclusion

It is evident from the two tests above that the compounds according to the invention are muscle-relaxing agents which are at least as effective as alverine, or even more effective, and which are useful in this regard in the smoothing of expression wrinkles and fine lines.

### Example 11

### Cosmetic Composition

This composition is prepared in a conventional manner for persons skilled in the art. The quantities indicated are percentages by weight.

| | |
|---|---|
| Compound 1 | 1% |
| Propylene glycol isostearate | 13% |
| Polyethylene glycol (8 EO) | 5% |
| Propylene glycol | 3% |
| Pentylene glycol | 3% |
| Glyceryl stearate and polyethylene | 5% |
| glycol stearate (100 EO) | |
| Oxyethylenated sorbitan monostearate (20 EO) | 0.5% |
| Oxyethylenated (20 EO) oxypropylenated (5 PO) | 1% |
| cetyl alcohol | |
| Gelling agents | 0.5% |
| C₁₂₋₁₅ alkyl benzoates | 4% |
| Ethanol | 3% |
| Sodium hydroxide | 0.12% |
| Preservatives | 0.7% |
| Water | qs 100% |

This fluid is intended to be used as once- or twice-daily applications to the face and the forehead in order to attenuate expression wrinkles and fine lines and to relax the lineaments of the face.

The above written description of the invention provides a manner and process of making and using it such that any person skilled in this art is enabled to make and use the same, this enablement being provided in particular for the subject matter of the appended claims, which make up a part of the original description.

As used above, the phrases “chosen from” and “selected from the group consisting of” include mixtures of the specified materials.

All references, patents, applications, tests, standards, documents, publications, brochures, texts, articles, etc. mentioned herein are incorporated herein by reference. Where a numerical limit or range is stated, all values and subranges therewithin are specifically included as if explicitly written out.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein.

1. A compound of formula (I): 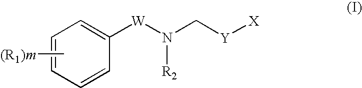
in which:
R₁ denotes a saturated or unsaturated, linear or branched C₁-C₈ alkyl group, or a group —CN, —OR₁₁, —SR₁₁, —NR₁₁R₁₂, —COR₁₁, —COOR₁₁, —CONR₁₁R₁₂, —NR₁₁—CO—R₁₂, —NR₁₁—CO—NR₁₂R₁₃ or —CF₃ or a halogen atom,
where R₁₁, R₁₂ and R₁₃ independently denote a hydrogen atom or a linear or branched C₁-C₄ alkyl group, or an aryl group optionally substituted with a group —OR, —NRR′, —COOR or CF₃,
where R and R′ independently denote a hydrogen atom or a linear or branched C₁-C₄ alkyl group,
R₂ denotes a hydrogen atom or an unsubstituted, saturated or unsaturated, linear or branched C₁-C₁₂ alkyl group,
W is an unsubstituted, linear C₂-C₄ alkylene or alkenylene chain,
X is a group —OR₁₁, or —NR₁₁R₁₂, where R₁₁ and R₁₂ have the meanings indicated above,
Y denotes an unsubstituted, linear or branched C₁₁-C₂₀ alkylene or alkenylene chain,
m is an integer between 0 and 5,
wherein when m is not zero, the groups R₁ may be identical or different,
addition salts with an acid thereof, and stereo-isomers thereof.

2. A compound according to claim 1, wherein the compound is a salt obtained by addition with an inorganic acid selected from the group consisting of hydrochloric, sulphuric, nitric and phosphoric acids.

3. A compound according to claim 1, wherein the compound is a salt obtained by addition with an organic acid selected from the group consisting of succinic, fumaric, lactic, glycolic, citric and tartaric acids.

4. A compound according to claim 1, wherein at least one of the following conditions is satisfied:
m=0 or 1,
R₁ is a group —OR₁₁, where R₁₁ is a hydrogen atom or a linear or branched C₁-C₄ alkyl group,
R₂ is a hydrogen atom or a saturated, linear or branched C₁-C₆ alkyl group,
W is an unsubstituted, linear C₂-C₄ alkylene or alkenylene chain,
X is a group —OR₁₁ where R₁₁ is a hydrogen atom or a linear or branched C₁-C₄ alkyl group,
Y is an unsubstituted, branched C₁₁-C₁₆ alkylene chain.

5. A compound according to claim 4, wherein at least one of the following conditions is satisfied:
m=0 or 1, R₁ is a group —OH or —OCH₃,
R₂ is a hydrogen atom or an ethyl group,
W is a trimethylene or propenylene chain,
X is a group —OH,
Y is an unsubstituted di(C₅-C₇)alkyl ethylene or di(C₅-C₇)dialkyl pentylene chain.

6. A compound according to claim 5, wherein: m=0; R₂ is an ethyl group; X═OH; Y is a dipentylethylene chain; and W is a trimethylene chain.

7. A composition suitable for topical application to the skin, comprising, in a physiologically acceptable medium, at least one compound of formula (I): 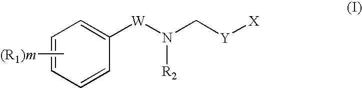
in which:
R₁ denotes a saturated or unsaturated, linear or branched C₁-C₈ alkyl group, or a group —CN, —OR₁₁, —SR₁₁, —NR₁₁R₁₂, —COR₁, —COOR₁₁, —CONR₁₁R₁₂, —NR₁₁—CO—R₁₂, —NR₁₁—CO—NR₁₂R₁₃ or —CF₃ or a halogen atom,
where R₁₁ R₁₂ and R₁₃ independently denote a hydrogen atom or a linear or branched C₁-C₄ alkyl group, or an aryl group optionally substituted with a group —OR, —NRR′, —COOR or CF₃,
where R and R′ independently denote a hydrogen atom or a linear or branched C₁-C₄ alkyl group,
R₂ denotes a hydrogen atom or an unsubstituted, saturated or unsaturated, linear or branched C₁-C₁₂ alkyl group,
W is an unsubstituted, linear C₂-C₄ alkylene or alkenylene chain, or a methylene chain, X is a group —OR₁₁ or —NR₁₁R₁₂, where R₁₁ and R₁₂ have the meanings indicated above,
Y denotes an unsubstituted, linear or branched C₁₁-C₂ alkylene or alkenylene chain,
m is an integer between 0 and 5,
wherein when m is not zero, the groups R₁ may be identical or different, addition salts with an acid thereof, and stereo-isomers thereof.

8. The composition according to claim 7, comprising a salt obtained by addition with an inorganic acid selected from the group consisting of hydrochloric, sulphuric, nitric and phosphoric acids.

9. The composition according to claim 7, comprising a salt obtained by addition with an organic acid selected from the group consisting of succinic, fumaric, lactic, glycolic, citric and tartaric acids.

10. The composition according to claim 7, wherein the compound of formula (I) is such that at least one of the following conditions is satisfied:
m=0 or 1,
R₁ is a group —OR₁₁ where R₁₁ is a hydrogen atom or a linear or branched C₁-C₄ alkyl group,
R₂ is a hydrogen atom or a saturated, linear or branched C₁-C₆ alkyl group,
W is an unsubstituted, linear C₂-C₄ alkylene or alkenylene chain,
X is a group —OR₁₁, where R₁₁ is a hydrogen atom or a linear or branched C₁-C₄ alkyl group,
Y is an unsubstituted, branched C₁₁-C₁₆ alkylene chain.

11. The composition according to claim 7, wherein the compound of formula (I) is such that at least one of the following conditions is satisfied:
m=0 or 1,
R₁ is a group —OH or —OCH₃,
R₂ is a hydrogen atom or an ethyl group,
W is a trimethylene or propenylene chain,
X is a group —OH,
Y is an unsubstituted di(C₅-C₇)alkyl ethylene or di(C₅-C₇)dialkyl pentylene chain.

12. The composition according to claim 11, wherein the compound of formula (I) is such that: m=0; R₂ is an ethyl group; X═OH; Y is a dipentylethylene chain; and W is a trimethylene chain.

13. The composition according to claim 7, wherein the compound of formula (I) represents from 0.1 to 2% of the total weight of the composition.

14. The composition according to claim 7, further comprising at least one compound selected from the group consisting of: desquamating agents; moisturizing agents; depigmenting or propigmenting agents; antiglycation agents; NO-synthase inhibitors; agents stimulating the synthesis of dermal or epidermal macromolecules and/or preventing their degradation; agents stimulating the proliferation of fibroblasts and/or of the keratinocytes or stimulating the differentiation of the keratinocytes; muscle-relaxing agents; tightening agents; antipollution and/or anti-radical agents; agents acting on the microcirculation; agents acting on the energy metabolism of the cells; and mixtures thereof.

15. A method, comprising topically applying to wrinkles and/or fine lines at least one compound of formula (I): 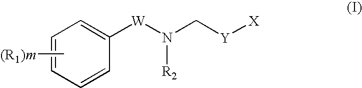
in which:
R₁ denotes a saturated or unsaturated, linear or branched C₁-C₈ alkyl group, or a group —CN, —OR₁₁, —SR₁₁, —NR₁₁R₁₂, —COR₁₁, —COOR₁₁, —CONR₁₁R₁₂, —NR₁₁, —CO—R₁₂, —NR₁₁—CO—NR₁₂R₁₃ or —CF₃ or a halogen atom,
where R₁₁, R₁₂ and R₁₃ independently denote a hydrogen atom or a linear or branched C₁-C₄ alkyl group, or an aryl group optionally substituted with a group —OR, —NRR′, —COOR or CF₃,
where R and R′ independently denote a hydrogen atom or a linear or branched C₁-C₄ alkyl group,
R₂ denotes a hydrogen atom or an unsubstituted, saturated or unsaturated, linear or branched C₁-C₁₂ alkyl group,
W is an unsubstituted, linear C₂-C₄ alkylene or alkenylene chain, or a methylene chain,
X is a group —OR₁₁ or —NR₁₁R₁₂, where R₁₁ and R₁₂ have the meanings indicated above,
Y denotes an unsubstituted, linear or branched C₁₁-C₂₀ alkylene or alkenylene chain,
m is an integer between 0 and 5,
wherein when m is not zero, the groups R₁ may be identical or different,
addition salts with an acid thereof, and stereo-isomers thereof.

16. The method according to claim 15, wherein the said wrinkles and fine lines are expression wrinkles and fine lines.

17. A method for the cosmetic treatment of wrinkled skin, comprising topically applying to the skin a composition according to claim 7.

18. The method according to claim 17, wherein said composition is applied to the areas of the face or of the forehead marked by expression wrinkles and fine lines, and/or to people having expression wrinkles and fine lines.

19. The method according to claim 17, wherein the said composition is applied to the wrinkles and fine lines arranged radially around the mouth and/or the eyes and/or horizontally on the forehead and/or situated in the inter-superciliary space.
